(19) United States
(12) Patent Application Publication
(10) Pub. No.: US 2016/0310505 A1
(43) Pub. Date: Oct. 27, 2016

(54) PROSTAMIDES FOR ENHANCEMENT OF LEPTIN PRODUCTION

(71) Applicant: Allergan, Inc., Irvine, CA (US)

(72) Inventors: Neil J. Poloso, Lake Forest, CA (US); David F. Woodward, Lake Forest, CA (US); Timothy J. Maziasz, Aliso Viejo, CA (US); Suzanne Kanaly, Trabuco Canyon, CA (US); Grantley Charles, Tustin, CA (US); Michael Garst, Newport Beach, CA (US); Robert M. Burk, Laguna Beach, CA (US)

(21) Appl. No.: 14/952,208

(22) Filed: Nov. 25, 2015

Related U.S. Application Data
(63) Continuation of application No. 14/209,099, filed on Mar. 13, 2014, now abandoned.
(60) Provisional application No. 61/793,132, filed on Mar. 15, 2013.

(51) Int. Cl. A61K 31/5578; A61K 9/70; A61K 9/00

(52) U.S. Cl. CPC A61K 31/5578; A61K 9/0014; A61K 9/0019; A61K 9/7023

(57) ABSTRACT

Prostamides such as bimatoprost and its pro-drugs for enhancement of leptin production and appetite suppression.

### CROSS REFERENCE TO RELATED APPLICATION

This application is a continuation of U.S. patent application Ser. No. 14/209,099, filed on Mar. 13, 2014, which in turn claims the benefit of U.S. Provisional Patent Application Ser. No. 61/793,132, filed Mar. 15, 2013, the disclosures of which are incorporated herein by reference in their entireties.

### FIELD OF THE INVENTION

The present invention is directed to the use of prostamides such as bimatoprost and its pro-drugs for the enhancement of leptin production and appetite suppression.

### BACKGROUND OF THE INVENTION

Leptin is major hormone produced in adipose tissue that has been shown to regulate appetite [Halaas J L, Gajiwala K S, Maffei M, et al. Weight-reducing effects of the plasma protein encoded by the obese gene. Science. 1995; 269 (5223):543-546r] and alter the taste for sweetness of food [Kawai K, Sugimoto K, Nakashima K, Miura H, Ninomiya Y, Proc Natl Acad Sci USA. 2000 Sep. 26; 97 (20):11044-9]. Leptin is also a mediator of long-term regulation of energy balance, suppressing food intake and thereby inducing weight loss (Klok, “The Role of Leptin and Ghrelin in the Regulation of Food Intake and Body Weight in Humans: A Review.” Obes. Rev. 2007, January; 8 (1): 21-34).

Bimatoprost (AGN 192024) is a synthetic prostamide which has been used in intraocular pressure lowering therapeutics such as LUMIGAN® 0.03, LUMIGAN® 0.01 and GANFORT®. Bimatoprost has also been shown to induce eyelash and hair growth and is marketed for that purpose with the commercial product LATISSE®. Bimatoprost applied topically has also been shown to result in subcutaneous fat loss at sites distant from the application site (see FIG. 1) in rats during a six month study of once a day topical application (˜10% body surface coverage). This application also led to a reduction in weight over time (see FIG. 2).

### SUMMARY OF THE INVENTION

It is hereby proposed that in addition to other therapeutic uses, bimatoprost can mediate weight loss and gain through modulation of the appetite suppressing hormone leptin. An additional benefit may be maintaining weight control in non-obese individuals, that is in suppressing appetite in individuals with normal weight, use in conjunction with or without dieting, or as an adjunct to bariatric surgery, gastric banding (Lap-band) or other methods where weight control would be suitable (e.g., prolonged systemic steroid use, during smoking cessation programs to alleviate over-eating, or intake of foods high in sugar). Further, the use of bimatoprost as described in the present application can be applied to a wide range of disorders such as metabolic disease, type II diabetes, insulin resistance syndrome and non-alcoholic fatty liver. The delivery of bimatoprost may be topical, oral, systemic such as by skin patch, subcutaneous, sublingual and by suppository to obtain systemic exposure of the compound.

The term “prodrug” is used according to its plain ordinary meaning and is intended to mean compounds that require a chemical or enzymatic transformation in order to release the active parent drug in vivo prior to producing a pharmacological effect.

A “pharmaceutically acceptable carrier” or “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The terms “treat” “treating” or “treatment” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer, in inhibiting its growth and or causing remission of cancer.

An “effective amount” of a compound is an amount sufficient to contribute to the treatment, prevention, or reduction of a symptom or symptoms of a disease. Where recited in reference to a disease treatment, an “effective amount” may also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) a disease, disorder or condition, or reducing the likelihood of the onset (or reoccurrence) of a disease, disorder or condition or symptoms thereof. The full prophylactic effect does not necessarily occur by administration of one dose, and may occur only after administration of a series of doses. Thus, a prophylactically effective amount may be administered in one or more administrations.

The term “topical” in the context of methods described herein relates in the customary sense to the administration of a compound or pharmaceutical composition which is incorporated into a suitable pharmaceutical carrier and administered at a topical treatment site of a subject. Accordingly, the term “topical pharmaceutical composition” includes those pharmaceutical forms in which the compound is administered externally by direct contact with a topical treatment site, e.g., the skin. The term “topical epidermal pharmaceutical composition” refers to a pharmaceutical composition suitable for administering directed to the epidermal layer of the skin, e.g., the palpebra, the supercilium, the scalp, or the body. The term “topical administering” refers to administering externally by direct contact with a topical treatment site. The term “topical epidermal administering” refers to administering externally by direct contact with the epidermis.

Some embodiments of the invention are included in the following paragraphs:
1) A method of enhancing leptin levels in a human comprising administering bimatoprost to the human.
2) The method of paragraph 1, wherein the bimatoprost is administered systemically.
3) The method of paragraph 1, wherein the bimatoprost is administered topically.
4) The method of paragraphs 1-3, wherein the bimatoprost enhances leptin production in adipose tissue.
5) The method of paragraphs 2-4, wherein the method enhances leptin production in pre-adipocytes.
6) The method of paragraphs 1-5, wherein the method suppresses appetite in a human.
7) The method of paragraphs 1 and 2, wherein the method is useful in treating type II diabetes.
8) The method of paragraphs 1 and 2, wherein the method is useful for treating a condition selected from the group consisting of metabolic disease, type II diabetes, insulin resistance syndrome, metabolic syndrome and non-alcoholic fatty liver.
9) The method of paragraphs 1 and 2, wherein bimatoprost is administered orally.
10) The method of paragraph 3, wherein bimatoprost is administered by a transdermal skin patch.
11) The method of paragraph 2, wherein bimatoprost is administered subcutaneously.
12) The method of paragraphs 1-6, wherein the method is useful in treating obesity.
13) The methods of paragraphs 1-6, wherein bimatoprost is in the form of a bimatoprost prodrug.
14) The method of paragraph 1, wherein the method reduces or prevents differentiation of pre-adipocytes than would occur if the bimatoprost was not administered.
15) The method of paragraph 1, wherein the topical or subcutaneous application of bimatoprost results in fat reduction at the application site and distal to the application site.
16) A method of losing weight or causing systemic weight loss comprising administering bimatoprost to a patient.
17) The method of paragraph 16, where the bimatoprost is applied topically.
18) The method of paragraphs 1-17, wherein the bimatoprost is 0.1-3% w/v.
19) The method of paragraph 1, wherein the method prevents the onset of non-alcoholic fatty liver.
20) The method of paragraphs 1 and 16, wherein the bimatoprost concentration is selected from one consisting of 0.1%, 0.3%, 1%, 2% and 3% w/w bimatoprost.
21) The method of paragraph 1, wherein the bimatoprost is administered to the patient in a transdermal skin patch.

### BRIEF DESCRIPTION OF THE DRAWINGS

FIG. 1 shows subcutaneous fat reduction at sites distal to the application site of bimatoprost with vehicle and application of 3% w/v bimatoprost;

FIG. 2 shows reduction in body weight after treatment with bimatoprost. Rats were treated topically with bimatoprost at doses shown in FIG. 2;

FIG. 3 shows that bimatoprost increases Leptin production in human pre-adipocytes. Vehicle is DMSO, bimatoprost treatment at 1 μM. Stimulation of leptin over 8-days of bimatoprost treatment;

FIG. 4 shows that bimatoprost results in elevated leptin levels of rats on a cafeteria diet; and,

FIG. 5 shows bimatoprost dose-dependently decreases cafeteria diet induced fatty liver changes.

### DETAILED DESCRIPTION OF THE INVENTION

The present invention covers a novel use of bimatoprost including other known prostamides, and structural analogs of bimatoprost and its pro-drugs (non-limiting examples include acyl, acyl esters, amino acids and phosphates and prostamides as disclosed in U.S. Pat. No. 5,688,819 which is herein incorporated by reference). Bimatoprost was examined for the effect on hormones released from adipose tissue.

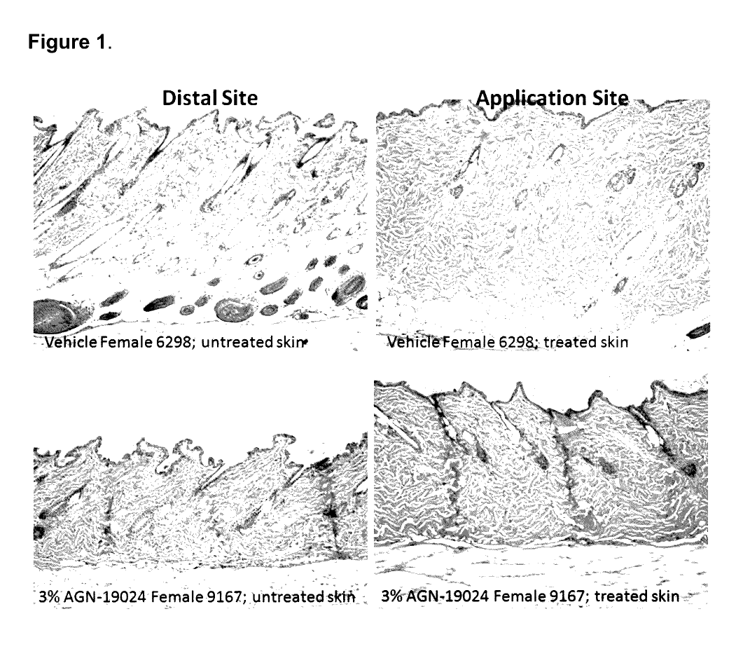

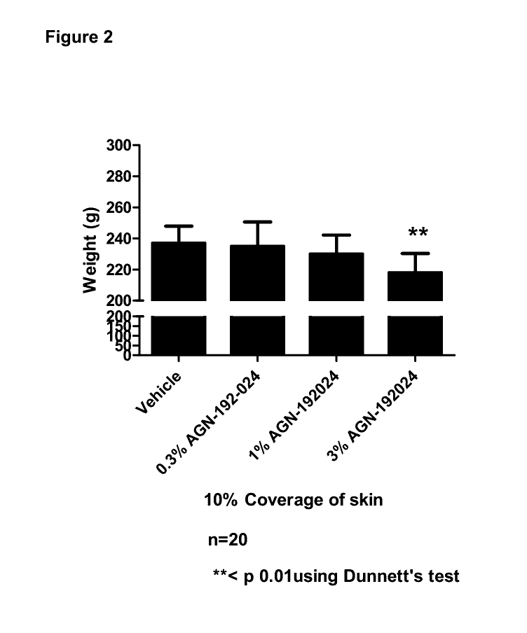

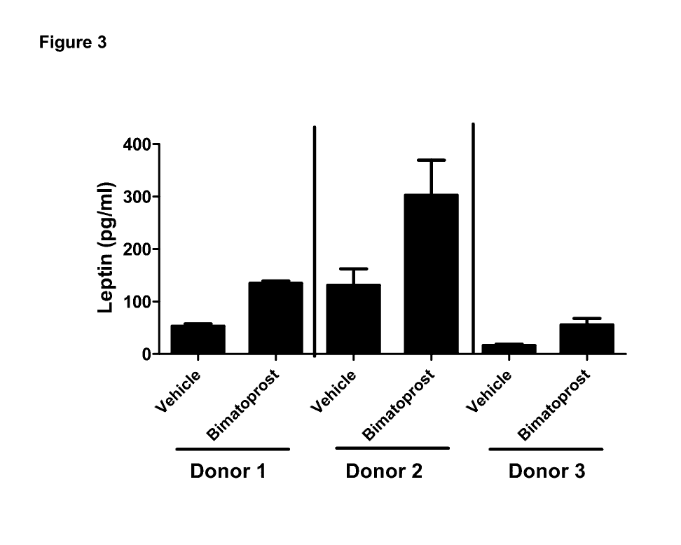

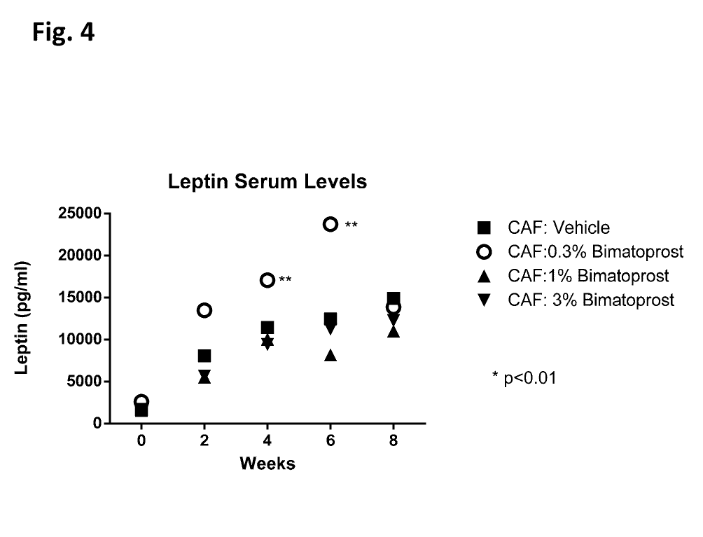

In FIG. 1, bimatoprost was applied topically once per day for 6 months to rats. Treatment of rats resulted in substantial local subcutaneous fat reduction, as well as reduction at adjunct and distal sites. FIG. 2 describes the systemic exposure (topically applied) of bimatoprost by measuring blood levels of the compound after treatment. A major target of bimatoprost for action is the pre-adipocyte, as determined by its activity to inhibit differentiation. In conjunction, treatment of human pre-adipocytes result in an increase in leptin production. It was also discovered that application of bimatoprost to pre-adipocytes, but not mature adipocytes, led to an increase in leptin levels in vitro (FIG. 3), a protein known to suppress appetite. The in vivo action of bimatoprost is likely a dual mechanism to inhibit food intake and suppress replenishment of fat cells during normal turnover. FIG. 4 shows male rats on a cafeteria diet (CAF) were treated with topical bimatoprost in BSHG formulation (0.3%, 1%, or 3%) or vehicle (see FIG. 2) daily. Blood was drawn every 2 weeks and the serum was analyzed for leptin levels by luminex assay. Male rats dosed with 0.3% bimatoprost showed elevated levels of Leptin (p<0.01, 2-way ANOVA). A cafeteria diet is a high sugar and fat diet with typical “junk food”:

TABLE I

| | | 2 | 3 | 4 | | 6 | | 8 | | |
|---|---|---|---|---|---|---|---|---|---|---|
| | 1 | Fat | Total | Total | 5 | Dietary | 7 | Sat. | 9 | 10 |
| Food Type | Kcal | (kcal) | Fat | Carb | Protein | fiber | Sugars | Fat | Chol. | Sodium |
| Standard Chow ® | 4.07 | 0.36 | 0.04 | 0.49 | 0.24 | 0.05 | 0 | 0 | 0 | 4 |
| Kellog's Fruit Loops ® | 4 | 0.33 | 0.03 | 0.87 | 0.03 | 0.03 | 0.43 | 0 | 0 | 4.7 |
| General Mills Coca Puffs ® | 4.07 | 0.37 | 0.06 | 0.85 | 0.04 | 0.04 | 0.44 | 0 | 0 | 5.6 |
| Little Debbie Fudge Rounds ® | 4.48 | 1.49 | 0.16 | 0.7 | 0.03 | 0.03 | 0.45 | 0.06 | 0.08 | 2.39 |
| Peanut butter cookies | 5 | 1.88 | 0.22 | 0.63 | 0.06 | 0 | 0.31 | 0.06 | 0 | 3.13 |
| Hershey Reeses Pieces ® | 5.13 | 2.05 | 0.23 | 0.62 | 0.13 | 0.03 | 0.54 | 0.18 | 0 | 2.05 |
| Hostess ® blueberry mini-muffins | 4.74 | 2.28 | 0.26 | 0.56 | 0.05 | 0.02 | 0.33 | 0.04 | 0.7 | 3.16 |
| Nestle Crunch ® | 4.86 | 2.29 | 0.26 | 0.69 | 0.06 | 0.03 | 0.51 | 0.14 | 0.14 | 0.86 |
| Cheez-it ® | 5.33 | 2.33 | 0.27 | 0.6 | 0.13 | 0.03 | 0.03 | 0.07 | 0 | 8.33 |
| General Mills Coca Puffs ® | 4.39 | 2.46 | 0.28 | 0.44 | 0.05 | 0.02 | 0.26 | 0.04 | 0.53 | 2.46 |
| Keebler TownHouse Butter Crackers ® | 5.36 | 2.5 | 0.29 | 0.61 | 0.07 | 0.04 | 0.04 | 0.05 | 0 | 6.43 |
| Sugar wafers | 5.16 | 2.58 | 0.29 | 0.65 | 0.03 | 0 | 0.48 | 0.06 | 0 | 0.65 |
| Kroger ® hot dog wieners | 3.33 | 2.67 | 0.29 | 0.09 | 0.11 | 0 | 0.04 | 0.1 | 0.78 | 10.22 |
| Kroger ® mild cheddar/jack cheese | 3.93 | 2.86 | 0.32 | 0.04 | 0.25 | 0 | 0 | 0.18 | 1.07 | 6.43 |
| Kroger ® wedding cakes | 5.67 | 3 | 0.33 | 0.57 | 0.03 | 0 | 0.33 | 0.07 | 0 | 2.5 |
| Frito-lay ® Lay wavy | 5.36 | 3.21 | 0.36 | 0.54 | 0.07 | 0.04 | 0 | 0.04 | 0 | 6.43 |
| Kroger ® pork rinds BBQ | 5.71 | 3.21 | 0.36 | 0 | 0.57 | 0 | 0 | 0.11 | 1.43 | 28.57 |
| Kroger ® pepperoni slices | 4.67 | 4 | 0.43 | 0 | 0.2 | 0 | 0 | 0.17 | 1 | 16.67 |

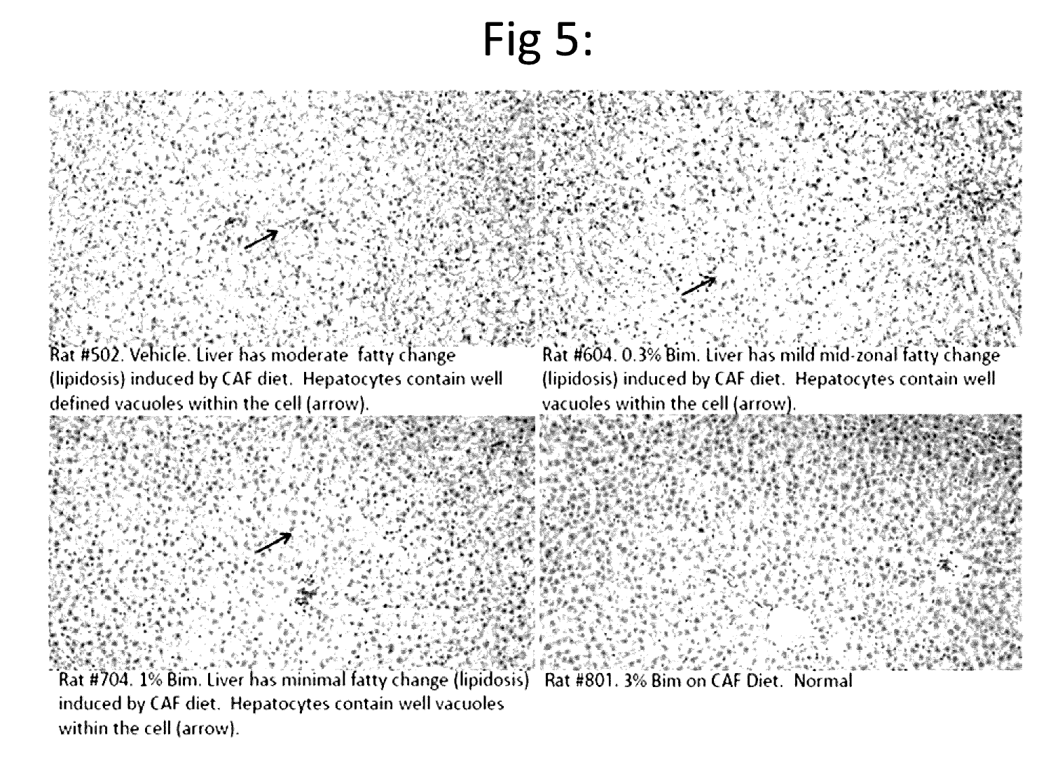

FIG. 5 shows that rats receiving 0.3% and 1% bimatoprost formulations had reduced lipidosis as compared to the control. Topical administration of bimatoprost inhibited cafeteria diet induced fatty liver disease. Rats were fed the cafeteria diet for 10 weeks and administered bimatoprost daily. At the end of 10 weeks, livers were resected and examined by histology. This result shows bimatoprost can inhibit lipid droplet deposition in the liver due to the excess dietary consumption of fats and sugar from the cafeteria diet. This has important consequences in the potential treatment of non-alcoholic fatty liver disease (NAFLD).

The compounds and pharmaceutical compositions disclosed herein can be prepared and administered in a variety of forms including a dermal or transdermal skin patch, a transdermal implant, cream, lotion, shampoo, solution, emulsion, gel, colloid, or foam. Accordingly, pharmaceutical compositions contemplated herein include a pharmaceutically acceptable carrier or excipient and one or more compounds described herein.

Pharmaceutical compositions contemplated herein may be prepared by combining a therapeutically effective amount of bimatoprost or another prostamide in combination with one or more pharmaceutically acceptable excipients. Pharmaceutical admixtures suitable for use in the present invention include those described in, for example, in PHARMACEUTICAL SCIENCES (17th Ed., Mack Pub. Co., Easton, Pa.) and WO 96/05309, the teachings of both of which are hereby incorporated by reference.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

TABLE 2
Some bimatoprost formulations include:

| | | Bimatoprost | Bimatoprost | Bimatoprost | Bimatoprost |
|---|---|---|---|---|---|
| | | 0.3% | 1.0% | 3.0% | 2.0% |
| | | (Propylene | (Propylene | (Propylene | (Propylene |
| Ingredient (% | | Glycol) | Glycol) | Glycol) | Glycol) |
| w/w) | Function | Solution | Solution | Solution | Solution |
| Bimatoprost | Active | 0.3 | 1.0 | 3.0 | 2.0 |
| Propylene glycol | Penetration | 10.0 | 10.0 | 10.0 | 10.0 |
| Diethylene glycol | enhancer | 10.0 | 10.0 | 10.0 | 10.0 |
| monoethyl ether | | | | | |
| Ethyl alcohol | | 30.0 | 30.0 | 30.0 | 30.0 |
| Glycerin | | 2.0 | 2.0 | 2.0 | 2.0 |
| Carbomer (Ultrez | Thickener | 0.15 | 0.15 | 0.15 | 0.15 |
| 10) | | | | | |
| Triethanolamine | Neutralizing | 0.16 | 0.16 | 0.16 | 0.16 |
| | agent | | | | |
| Purified water | Vehicle | 47.66 | 47.59 | 47.39 | 47.49 |

TABLE 3
Example Components with Function and Concentration Ranges

| | | Composition |
|---|---|---|
| Ingredient | Function | (% w/w) |
| bimatoprost | Active | 0.03-5.0 |
| carbomer | Thickener | 0.05-1.0 |
| base | Neutralizing Agent | 0.01-2.0 |
| ethanol | Penetration enhancers | 10-90 |
| glycerin | | 1.0-20 |
| diethylene glycol monoethyl ether | | 1.0-50 |
| propylene glycol | | 1-50 |
| polysorbate 20 | | 0.1-5.0 |
| polysorbate 40 | | 0.1-5.0 |
| polysorbate 60 | | 0.1-5.0 |
| polysorbate 80 | | 0.1-5.0 |
| PPG-5 ceteth-20 | | 0.1-5.0 |
| oleic acid | | 0.1-5.0 |
| isostearyl isostearate | | 0.1-10 |
| isopropyl myristate | | 0.1-10 |
| dipropylene glycol dimethyl ether | | 1-50 |
| diethylene glycol | | 1-50 |
| dipropylene glycol | | 1-50 |
| caprylic/capric triglycerides | | 0.1-10 |
| benzyl alcohol | Preservative | 0.1-2.0 |
| silicone | Occlusive Agent | 0.1-10 |
| water | Vehicle | 0-90 |

Bimatoprost or another prostamide can be included in compositions of the embodiments disclosed herein in an amount of between 0.0001 and 15% (w/v), between 0.0001 and 10% (w/v), between 0.0001 and 5% (w/v), between 0.0005 and 3% (w/v), between 0.00075 and 2% (w/v), between 0.001 and 1.0% (w/v), between 0.001 and 0.1 (w/v), between 0.005 and 0.05% (w/v), or 0.01% (w/v) of the composition. In some embodiments, an amount of the active compound such as bimatoprost or another prostamide is 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, 8.0%, 9% and 10% w/w.

In some embodiments, an effective amount, e.g., a therapeutically effective amount, of the active compound in a pharmaceutical composition is afforded at a concentration of about 1×10⁻⁷ to 50% (w/w), about 0.001 to 50% (w/w), about 0.01 to 50% (w/w), about 0.1 to 50% (w/w), or about 1 to 50% (w/w). In some embodiments, the therapeutically effective amount of the active compound such as bimatoprost or another prostamide in a pharmaceutical composition is 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9% and 1.0%, 1.1%, 1.2%, 1.3%, 1.4%, 1.5%, 1.6%, 1.7%, 1.8%, 1.9%, 2.0%, 3.0%, 4.0% and 5.0% w/w.

### EXAMPLE 1

### Use of Bimatoprost Patch for Enhancing Leptin Production and Weight Loss

A 51 year old Caucasian male who is morbidly obese applies a bimatoprost transdermal skin patch on his arm, which uniformly releases a 5% w/w bimatoprost formulation over a thirty day period. During the thirty-day period, the patient's leptin levels increase leading to suppressed appetite and weight loss. The patient loses 6 pounds more than he would have otherwise lost without using the bimatoprost transdermal skin patch.

### EXAMPLE 2

### Use of Topical Bimatoprost to Maintain Weight

A 43 year old Hispanic female applies a 3% w/w bimatoprost gel to her skin once a day. After several days, the 43 year old Hispanic female experiences elevated leptin levels which suppresses appetite. Over a sixty (60) day period, the patient maintains her weight through appetite suppression.

### EXAMPLE 3

### Use of a Bimatoprost Patch to Control Glucose Levels in a Prediabetes Patient

A 61 year old African-American male with elevated blood pressure has been determined by doctors to have prediabetes. In addition to being on a low fat, low sugar diet, the patient uses a transdermal bimatoprost patch which releases a 3% w/w bimatoprost formulation through the dermis and into the blood stream. The patient experiences an immediate increase in blood leptin levels and a reduction in appetite and experiences weight loss while using the bimatoprost patch.

### EXAMPLE 4

### Use of Topically Delivered Bimatoprost to Treat Non-Alcoholic Fatty Liver

A 70 year old Caucasian male is diagnosed with non-alcoholic fatty liver. The patient applies a transdermal bimatoprost patch which releases a 2% w/w bimatoprost formulation. The patient experiences a reduction in lipidosis in the liver that would have occurred had the patient not been administered bimatoprost.

### EXAMPLE 5

### Use of Topically Delivered Bimatoprost in Dieting

A healthy 27 year old Caucasian female in an effort to lose weight is on a low fat diet. In order to suppress her appetite, the 27 year old Caucasian female applies a bimatoprost transdermal patch which continually releases 1% w/w bimatoprost for 30 days. As a result, her leptin levels rise and she experiences suppression of her appetite and greater weight loss in comparison to had she not applied the transdermal patch with bimatoprost.

1) A method of enhancing leptin levels in a human comprising administering bimatoprost to the human.

2) The method of claim 1, wherein the bimatoprost is administered systemically.

3) The method of claim 1, wherein the bimatoprost is administered topically.

4) The method of claim 3, wherein the bimatoprost enhances leptin production in adipose tissue.

5) The method of claim 3, wherein the method enhances leptin production in pre-adipocytes.

6) The method of claim 1, wherein the method suppresses appetite in a human.

7) The method of claim 3, wherein the method is useful in treating type II diabetes.

8) The method of claim 3, wherein the method is useful for treating a condition selected from the group consisting of metabolic disease, type II diabetes, insulin resistance syndrome, metabolic syndrome and non-alcoholic fatty liver.

9) The method of claim 1, wherein the method results in weight loss.

10) The method of claim 3, wherein bimatoprost is administered by a transdermal skin patch.

11) The method of claim 2, wherein bimatoprost is administered subcutaneously.

12) The method of claim 1, wherein the method is useful in treating obesity.

13) The methods of claim 1, wherein bimatoprost is a bimatoprost prodrug.

14) The method of claim 1, wherein the method reduces or prevents differentiation of pre-adipocytes.

15) The method of claim 1, wherein the topical or subcutaneous application of bimatoprost results in fat reduction at the application site and distal to the application site.

16) The method of claim 1, wherein the method prevents and reduces the onset of non-alcoholic fatty liver.

17) The method of claim 1, wherein the bimatoprost concentration is selected from one consisting of 0.1%, 0.3%, 1%, 2% and 3% w/w bimatoprost.

18) The method of claim 1, wherein the bimatoprost is administered to the patient in a transdermal patch.
